SEQUENCE LISTING (1 sequence)

SEQ ID NO: 1 - protein, 27 aa, Limulus polyphemus
HAEHKVKIKVKQKYGQFPQGTEVTYTC

(19) United States
(12) Patent Application Publication
(10) Pub. No.: US 2023/0347037 A1
(43) Pub. Date: Nov. 2, 2023

(54) MATRICES FOR SELECTIVE BINDING OF AT LEAST ONE COMPONENT FROM A BODY FLUID

(71) Applicant: Alteco Medical AB, Lund (SE)

(72) Inventor: Lennart Wilhelm Ljunggren, LUND (SE)

(73) Assignee: Alteco Medical AB, Lund (SE)

(21) Appl. No.: 18/029,561

(22) PCT Filed: Sep. 28, 2021
(86) PCT No.: PCT/SE2021/050944

§ 371 Date: Mar. 30, 2023

(30) Foreign Application Priority Data
Oct. 1, 2020 (SE) 2051149-9

(51) Int. Cl. A61M 1/36

(52) U.S. Cl. CPC A61M 1/3679; A61M 2202/0413; A61M 2202/0042

(57) ABSTRACT

A matrix for selective binding of at least one component from a body fluid. The matrix may include a porous structure, a linker, a peptide covalently attached via the linker, and a coating of a polyalcohol.

### CROSS-REFERENCE TO RELATED APPLICATIONS

This application claims priority to International Patent Application No. PCT/SE2021/050944, filed Sep. 28, 2021, and Swedish Patent Application No. SE 2051149-9, filed on Oct. 1, 2020, the contents of both of which are hereby incorporated by reference in their entirety.

### TECHNICAL FIELD

The present disclosure relates to matrices for selective binding of at least one component from a body fluid. More specifically the present disclosure relates to matrices for the selective binding of lipopolysaccharide (LPS). The present disclosure also relates to a method of manufacturing such matrices, to methods for selectively binding and separating at least one component from a body fluid and to a device used in such methods as well as to the use of such matrices.

### BACKGROUND

Inflammatory processes, such as sepsis, are a major cause of morbidity and mortality in humans. It is estimated that, yearly, 400 000 to 500 000 episodes of sepsis result in 100 000 to 175 000 human deaths in the U.S. alone. In Germany, sepsis rates of up to 19% of patients stationed at intensive care units have been noted. Sepsis has also become the leading cause of death in intensive care units among patients with non-traumatic illnesses. Despite the major advances of the past decades in the treatment of serious infections, the incidence and mortality due to sepsis continues to rise.

There are three major types of sepsis characterized by the type of infecting organism. Gram-negative sepsis is the most common. The majority of these infections are caused by Escherichia coli, Klebsiella pneumoniae and Pseudomonas aeruginosa. Gram-positive pathogens, such as the staphylococci and the streptococci, are the second major cause of sepsis. The third major cause of sepsis are fungal infections, which constitute a relatively small percentage of the sepsis cases.

A well-established mechanism in sepsis is related to a toxic component of Gram-negative bacteria, the lipopolysaccharide (LPS, endotoxin) cell wall structure, which is composed of a fatty acid group, a phosphate group, and a carbohydrate chain.

Several of the host responses to LPS have been identified, such as release of cytokines, which are produced locally. In case of an extensive stimulation, however, there is a spill over to the peripheral blood and potential harmful effects are obtained, such as induced organ dysfunction.

The key mediators of septic shock are Tumor Necrosis Factor (TNF-α), Interleukine 1 (I1-1) and Interleukine 17 (I1-17), which are released by monocytes and macrophages. They act synergistically causing a cascade of physiological changes leading to circulation collapse and multi organ failure.

Antibiotics of varying types are widely used to prevent and treat infections. However, for many commonly used antibiotics an antibiotic resistance has developed among various species of bacteria. This is particularly true for the microbial flora resident in hospitals, where organisms are under a constant selective pressure to develop resistance. Furthermore, in hospitals, the high density of potentially infected patients and the extent of staff-to-staff and staff-to-patient contact facilitate the spread of antibiotic resistant organisms. Antibiotics can be toxic to varying degrees by causing allergy, interactions with other drugs, and causing direct damage to major organs (e.g. liver and kidney). Many antibiotics also change the normal intestinal flora, which can cause diarrhea and nutritional malabsorption.

Certain antibiotics are known to neutralize the action of endotoxins, such as polymyxin B. This antibiotic binds to the lipid A part of endotoxin and neutralizes its activity. Polymyxin B is not used routinely due to its toxicity, but is only given to patients under constant supervision and monitoring of the renal function.

In attempts to remove components from blood, different adsorbent materials have been prepared. An endotoxin removal adsorbent comprising a ligand immobilized on a solid phase support medium is shown in WO 01/23413. A preferred support medium is in the form of beads. When packed in a separation device, the solid phase support medium is porous enough to allow passage of blood cells between the beads.

Likewise, in WO 01/23413 the porous support material for endotoxin removal is beads, which can be filled into a container, the beads having a size sufficient to provide the required space between the beads when packed into a column or filter bed. The porous support material can also be microfiltration hollow-fibers or flat sheet membranes in order to minimize pressure drops.

EP 1 497 025 B1 discloses a method for selectively binding and separating at least one component from a body fluid without the need of separating blood into plasma and blood cells. The component may be LPS. The body fluid is passed through a rigid integral separation matrix whereby the component binds to at least one functional group in the matrix.

An object of the present disclosure is to provide an improved matrix for selective binding and separating at least one component from whole blood or body fluids.

### OVERVIEW

According to a first aspect, the above and other objects of the disclosure are achieved, in full or at least in part, by a matrix as defined by the claims. According to this claim the above object is achieved by a matrix for selective binding of at least one component from a body fluid, the matrix having a porous structure; wherein a peptide is covalently attached to the matrix via a linker; and wherein the matrix is coated with a polyalcohol.

According to a second aspect, the above and other objects of the disclosure are achieved, in full or at least in part, by a matrix for selective binding of at least one component from a body fluid, the matrix having a porous structure; wherein a peptide is covalently attached to the matrix via a linker; wherein the linker is covalently bound to a residue of an amino-group present in the matrix and covalently bound to a residue of a thiol-group present in the peptide; and wherein the linker is a heterobifunctional cross-linker chosen from the group consisting of succinimidyl 4-(N-maleimidomethyl)cyclohexane-1-carboxylate (SMCC), sulfo-succinimidyl 4-(N-maleimidomethyl)cyclohexane-1-carboxylate (Sulfo-SMCC), 4-((4-(cyanoethynyl)benzoyl)oxy)-2,3,5,6-tetrafluorobenzenesulfonate (CBTF), sulfo-4-((4-(cyanoethynyl)benzoyl)oxy)-2,3,5,6-tetrafluorobenzenesulfonate (Sulfo-CBTF), maleimide-poly(ethylene glycol)-succinimidyl ester, poly(ethylene glycol)-diglycidyl ether and 1-4-butanediol diglycidyl ether.

According to a third aspect, the above and other objects of the disclosure are achieved, in full or at least in part, by a matrix for selective binding of lipopolysaccharide (LPS) from a body fluid, the matrix having a porous structure; wherein a peptide according to SEQ ID NO 1 or a peptide having at least 80%, 85%, 90%, 95% or 99% homology with the peptide according to SEQ ID NO 1 is covalently attached to the matrix via a linker; wherein the linker is covalently bound to a residue of an amino-group present in the matrix and covalently bound to a residue of a thiol-group present in the peptide.

According to a fourth aspect, a matrix according to the present disclosure is manufactured by a method comprising the steps of a) providing a matrix comprising primary amino-groups; b) covalently attaching a peptide to the matrix via a linker to provide a matrix comprising a covalently attached peptide; c) adding a polyalcohol to the matrix comprising a covalently attached peptide; and d) subjecting the matrix obtained after step c) to irradiation.

According to a fifth aspect, a method for selectively binding and separating at least one component from a body fluid is provided. The method comprises the step of passing a body fluid through a matrix according to the present disclosure or through a matrix manufactured by the method according to the present disclosure, whereby said at least one component binds to the peptide bound covalently bound to the matrix.

According to a sixth aspect, a device for selective binding and separation of at least one component from a body fluid according to the method of the present disclosure is provided. The device comprises a housing, an inlet, an outlet and a first matrix, wherein the first matrix is a matrix according to the present disclosure or a matrix manufactured by the method according to the present disclosure.

According to a seventh aspect, a use of a matrix according to the present disclosure, of a matrix manufactured by the method according to the present disclosure or of a device according to the present disclosure for selectively binding and separating at least one component from a body fluid.

Other objectives, features and advantages of the present disclosure will appear from the following detailed description, from the drawings, as well as from the attached claims. It is noted that the disclosure relates to all possible combination of features.

Generally, all terms used in the claims are to be interpreted according to their ordinary meaning in the technical field, unless explicitly defined otherwise herein. All references to “a/an/the [element, device, component, means, step, etc.]” are to be interpreted openly as referring to at least one instance of said element, device, component, means, step, etc., unless explicitly stated otherwise. The steps of any method disclosed herein do not have to be performed in the exact order disclosed, unless explicitly stated.

As used herein, the term “comprising” and variations of that term are not intended to exclude other additives, components, integers or steps.

### BRIEF DESCRIPTION OF THE DRAWINGS

By way of example, embodiments of the present disclosure will now be described with reference to the accompanying drawings, in which:

FIG. 1 is a schematic drawing of a device according to the present disclosure.

### DETAILED DESCRIPTION

The present disclosure relates to matrices which are able to selectively bind at least one component from a body fluid. The present disclosure also relates to a method of manufacturing such matrix and to a method for selectively binding and separating at least one component from a body fluid by passing a body fluid through a matrix according to the present disclosure, as well as to a device for selectively binding and separating at least one component from a body fluid. The present disclosure also relates to the use of a matrix according to the present disclosure, of a matrix manufactured by the method according to the present disclosure or of a device according to the present disclosure for selectively binding and separating at least one component from a body fluid.

Specific aspects and embodiments of the present disclosure will be described in detail below.

Matrices

The matrices disclosed herein are for selective binding of at least one component from a body fluid.

General features applicable to the matrices disclosed herein are described below.

The matrices according to the present disclosure have a porous structure and a peptide is covalently attached to the matrix via a linker, wherein the linker is covalently bound to a residue of an amino-group present in the matrix and covalently bound to a residue of a thiol-group present in the peptide.

The body fluid may be whole blood, plasma or cerebrospinal fluid.

Preferably, the body fluid is whole blood.

The component, which is to be selectively bound, may be an endogenous component, i.e. a component produced by the patient whose body fluid, such as whole blood, is to be passed through the matrix.

The component, which is to be selectively bound, may be an exogenous component, i.e. a component which is not produced by the patient whose body fluid, such as whole blood, is to be passed through the matrix. Examples of such components may be a toxic component produced by infectious agent, such as from a bacterium, a virus or a fungus.

More specifically, the component is derived from a bacterium.

A specific example of a component, which is to be bound by a matrix disclosed herein, is lipopolysaccharide (LPS) produced by Gram-negative bacteria.

Thus, preferred embodiments of the disclosure relate to the selective binding of LPS and to the separation of toxic LPS from the body fluid, such as from the whole blood, of a patient suffering from sepsis caused by Gram-negative bacteria.

The matrices disclosed herein have a porous structure.

The pore size preferably ranges from 1 μm to 500 μm in diameter, more preferably from 70 μm to 170 μm, most preferred from 80 μm to 100 μm. Such pore sizes enables that high flow rates of whole blood may be maintained without cellular damage or cellular exclusion. When the body fluid does not contain any blood cells, the pore size may be 1 μm to 25 μm.

The matrices preferably have an active surface ranging from 0.5 cm² to 10 m², preferably 4 cm² to 6 m², as measured by the BET-method, measured either by nitrogen adsorption or mercury intrusion.

The matrices disclosed herein are preferably obtained by a process selected from the group comprising sintering, moulding and foaming processes.

Most preferably, a matrix as disclosed herein is obtained by a sintering process.

The matrices disclosed herein are preferably rigid integral matrices. As used herein, the term “rigid” means that a matrix is not flexible, not bendable or yielding, but able to withstand a pressure of at least 0.5 bar. The term “integral” means that a matrix with high surface area is an entire entity.

The porous structure of the matrix may be made of metal, inorganic oxide, carbon, glass, ceramic, synthetic polymer, and/or natural polymer, or mixtures thereof.

Porous solid metal structures with well-defined pore sizes and high surface areas can be manufactured by using strictly controlled sintering processes that produces uniformly-sized pores.

Different polymers may be produced as a moulded or extruded porous material with a porous structure, having the desired pore size as well a high surface area for the matrix. Alternatively, polymers may be produced as foam or as cryogel.

A wide variety of metals and alloys may be used, such as stainless steel, nickel, titanium, monel, inconel, hastelloy and other special metal materials. High surface area inorganic oxides, especially alumina and zirconia, may be used.

Sintered glass having adequate pore sizes may also be used.

Other natural rigid materials, such as amorphous silica, e.g. zeolites, and lava rock, may be used.

Natural materials and hybrids thereof, such as polysaccharides, e.g. cellulose, and other polymeric carbohydrate materials, may be used. Other suitable natural polymeric materials are polyamino acids, also those involving synthetic amino acids, polylactic acid, polyglycolic acid and its copolymers with lactic acid. In this connection the term “hybrid” encompasses derivatives of such natural materials, for example cellulose diacetate, which is a preferred polysaccharide derivative.

Suitable synthetic polymers are polyolefines, such as polyethylene, polypropylene, polybutylene, polymetylpentene, and ethylene vinyl acetate copolymers; vinylic polymers, such as polyvinyl alcohol, polyvinyl acetals, and polyvinylpyrrolidone; fluorine containing polymers, such as polytetrafluoroethylene, fluorinated ethylene-propylene copolymer, polychloroflouroethylene, polyvinylfluoride, and polyvinylidene fluoride; polyacrylates, such as polymethylmethacrylate, cyanoacrylate, polyacrylonitrile, and polymetacrylates; polyamides, such as polyacrylamide; polyimides, such as polyethylenimines; polystyrene and its copolymers, such as polystyrene and acrylonitrile-butadiene-styrene-polymers; silicone rubbers; polyesters/ethers; polycarbonates; polyurethanes; polysulfonates; polyglycols; polyalkydeoxides such as polyethyleneoxide, polypropyleneoxide; and copolymers or hybrids or mixtures thereof.

Other examples of suitable synthetic polymers are cyclic olefins and copolymers thereof.

Preferably, the matrices according to the present disclosure are synthetic polymers, more preferably polyolefins, such as polyethylene or polypropylene or mixtures thereof.

Especially, sintered synthetic polymers, such as sintered polyolefins, such as polyethylene or polypropylene or mixtures thereof, are preferred.

Most preferably, a matrix according to the present disclosure is sintered polyethylene. The sintered polyethylene has a porous structure. Preferably, the pore size ranges from 1 μm to 500 μm in diameter, more preferably from 70 μm to 170 μm, most preferred from 80 μm to 100 μm. When the body fluid does not contain any blood cells, the pore size may be 1 μm to 25 μm. Preferably, the active surface of a matrix according to the present disclosure ranges from 0.5 cm² to 10 m², preferably 4 cm² to 6 m², as measured by the BET-method, measured either by nitrogen adsorption or mercury intrusion.

The matrices described herein comprise a linker covalently bound to a residue of an amino-group present in the matrix. The amino-group may be part of the material used to produce the matrix or may be added by functionalization of the material used to produce the matrix.

It is possible to treat organic polymeric surfaces in NH₃ or allylamine in plasma environments to introduce amino-groups in the material.

Polymerization of bifunctional monomers of acrylic or allylic double bonds with polar groups as CHO, OH, NH₂, CN and COOH may be used to produce plasma polymers with high density of the functional groups. For example, surface functionalization of the inorganic and organic surfaces may be carried out in a plasma environment of allyl compounds, such as allylamine This gives a polymer surface having amino-groups bound to the polymeric surface via two covalent bonds, which are more stable than amino-groups bound to the polymeric surface via only one covalent bond.

Thus, in certain preferred embodiments, the polymeric surface is functionalized using allylamine In these cases, the functionalization functions as an extra linker, presenting the peptide to the environment in a favourable way.

Many of the above-mentioned polymers, especially those without functional groups, such as polyethylene, polypropylene, polytetrafluoroethylene etc., need a further treatment in order to alter their surface properties. Thus, a plasma or corona treatment, as mentioned above, of the polymer surface may be used to generate amino-groups, which are covalently attached to the surface of the polymer.

The coating may also be accomplished by means of a polymeric substance having functional groups. Examples of such substances are polylysine and polyarginine. This may be accomplished by e.g. covalent coupling or cross-linking using either functionalized matrix surface heparin, chitosan or polyethyleneimine Such a functionalization increases the number of free amino-groups and thus the amount of bound peptide may be increased.

Polyethyleneimine, heparin, polylysine or polyarginine can be covalently attached to the plasma-polymerized surface, e.g. either directly to the modified surface by reductive amination or by the use of bi- or tri-functional cross-linkers etc.. Such modifications increase the number of functional groups that can be used for coupling the peptide.

As explained above, a linker is covalently bound to a residue of an amino-group present in the matrix. The linker may be a homobifunctional cross-linker or a heterobifunctional cross-linker.

When the linker is a homobifunctional cross-linker, the linker is covalently bound to a residue of an amino-group in the matrix and to an amino-group present in the peptide. Examples of such homobifunctional cross-linkers include glutardialdehyde and diepoxides such as poly(ethylene glycol) diglycidyl ether and 1-4-butanediol diglycidyl ether.

Poly(ethylene glycol) diglycidyl ether and 1-4-butanediol diglycidyl ether bind selectively to amino-groups at a pH-value of about 11, and to tiol-groups at a pH-value of about 8 to 9. Thus, by controlling the pH, the nature of the group to which these linkers bind, may be controlled.

In poly(ethylene glycol)-diglycidyl ether, the poly(ethylene glycol)-moiety preferably comprises 2 to 20, such as 2 to 15, such as 2 to 8 poly(ethylene glycol)-moieties. The poly(ethylene glycol)-diglycidyl ether may have a molecular weight of 200 to 2000 g/mol.

Preferably, the linker is a heterobifunctional cross-linker. Such linkers bind to two different functional groups. When the linker is a heterobifunctional cross-linker, the linker is covalently bound to a residue of an amino-group in the matrix and to a thiol-group present in the peptide. By binding to two different functional groups, the risk of the improper binding of the peptide is reduced.

Preferably, the linker is a heterobifunctional cross-linker chosen from the group consisting of succinimidyl 4-(N-maleimidomethyl)cyclohexane-1-carboxylate (SMCC), sulfo-succinimidyl 4-(N-maleimidomethyl)cyclohexane-1-carboxylate (Sulfo-SMCC), 4-((4-(cyanoethynyl)benzoyl)oxy)-2,3,5,6-tetrafluorobenzenesulfonate (CBTF), sulfo-4-((4-(cyanoethynyl)benzoyl)oxy)-2,3,5,6-tetrafluorobenzenesulfonate (Sulfo-CBTF), maleimide-poly(ethylene glycol)-succinimidyl ester, poly(ethylene glycol)-diglycidyl ether and 1-4-butanediol diglycidyl ether.

Such linkers have been shown to bind the peptide in a stable manner to the matrix.

Since the linker binds to two different functional groups, the risk of the improper binding of the peptide is reduced.

The linkers disclosed herein act as coupling agents, coupling the peptide to the matrix. In addition, the linkers also provide a means to create a distance between the matrix and the peptide, such that the peptide is presented to the component that is to be bound by the matrix. If the peptide is too close to the surface of the matrix, possible available sites of interaction with the component are limited. Thus, by creating a distance between the matrix and the peptide, the availability of the peptide to the components present in the body fluid is increased, thereby increasing the number of binding sites for the component in the matrix. Preferably, the linker creates a distance from the surface of the matrix to the peptide of 6 carbon atoms or more.

In maleimide-poly(ethylene glycol)-succinimidyl ester, the poly(ethylene glycol)-moiety preferably comprises 2 to 20, such as 2 to 15, such as 2 to 8 poly(ethylene glycol)-moieties.

Poly(ethylene glycol) diglycidyl ether and 1-4-butanediol diglycidyl ether bind selectively to amino-groups at a pH-value of about 11, and to tiol-groups at a pH-value of about 8 to 9. Thus, by controlling the pH, the nature of the group to which these linkers bind, may be controlled.

In poly(ethylene glycol)-diglycidyl ether, the poly(ethylene glycol)-moiety preferably comprises 2 to 20, such as 2 to 15, such as 2 to 8 poly(ethylene glycol)-moieties. The poly(ethylene glycol)-diglycidyl ether may have a molecular weight of 200 to 2000 g/mol.

Preferably, the linker is SMCC, CBTF, maleimide-poly(ethylene glycol)-succinimidyl ester, poly(ethylene glycol)-diglycidyl ether or 1-4-butanediol diglycidyl ether.

Most preferably, the linker is SMCC or CBTF.

The peptide is a peptide capable of binding to a specific component present in the body fluid. One example of such specific components is a specific bacterial components, e.g. LPS.

When the component to be bound by the matrix is LPS, the peptide is an LPS -binding peptide. Preferably, the LPS-binding peptide is 4 to 40 amino acids long, more preferably 10 to 35 amino acids long, and most preferably 20 to 30 amino acids long. More preferably, the LPS-binding peptide is a peptide according to SEQ ID NO 1 or a peptide having at least 80%, 85%, 90%, 95% or 99% homology with the peptide according to SEQ ID NO 1.

The matrices of the present disclosure may be coated with a polyalcohol. Such a coating stabilizes the peptide. This is especially advantageous when a matrix according to the present disclosure is stored under dry conditions. The polyalcohol acts as a humectant, thus stabilizing the peptide. Moreover, the polyalcohol may act as a bacteriostatic compound, preventing the growth of bacteria in the matrix during the production before the matrix is sterilized. In other words, the polyalcohol prevents an increased bioburden.

Furthermore, if the matrix is made of a partly hydrophobic material, such as e.g. polyethylene, hydrophobic parts of the peptide may interact with hydrophobic parts of the matrix material and thus loose its ability to bind to the component, which is to be bound by the matrix. By coating the matrix, i.e. providing a conjugated matrix, with a polyalcohol, hydrophobic parts of the matrix material are made more hydrophilic, since hydrophobic parts of the polyalcohol interact with the hydrophobic parts of the matrix material and the hydrophilic parts of the polyalcohol are faced towards the peptide, thereby hindering the hydrophobic interaction between the peptide and the matrix material.

Before the matrix is used, it is rinsed with a physiological NaCl-solution to remove the polyalcohol. It is advantageous if the polyalcohol is biocompatible, i.e. non-toxic, in the event smaller residual amounts of the polyalcohol are left after rinsing.

Examples of biocompatible polyalcohols, which may be used, are propane-1,2,3-triol, glucose, trehalose and a mixture thereof. Preferably, the polyalcohol is propane-1,2,3-triol, also known as glycerol or glycerin(e). Propane-1,2,3-triol is an endogenous non-toxic compound, making it especially suitable for coating of the matrix. Furthermore, it is a liquid at room temperature and is thus easy to handle. Further, since it is a liquid it will not evaporate or crystallize and is thus an especially effective humectant.

Another advantage of the presence of a polyalcohol-coating is that such a coating acts as a radical-scavenger, thereby protecting the matrix from beta- and gamma-radiation, which may be used for sterilizing the matrix.

Preferably, the polyalcohol is used in an amount corresponding to up to 0.1 to 0.5 g/m² of the matrix.

In the following, examples of matrices according to the present disclosure will be described. Effects and advantages of specific features are, unless stated otherwise, as describe above in the general section.

Matrix A has a porous structure. A peptide is covalently attached to the matrix via a linker; wherein the matrix is coated with a polyalcohol.

The polyalcohol coating stabilizes the peptide. This is especially advantageous when a matrix according to the present disclosure is stored under dry conditions. The polyalcohol acts as a humectant, thus stabilizing the peptide. Moreover, the polyalcohol may act as a bacteriostatic compound, preventing the growth of bacteria in the matrix during the production before the matrix is sterilized. In other words, the polyalcohol prevents an increased bioburden.

Furthermore, if the matrix is made of a partly hydrophobic material, such as e.g. polyethylene, hydrophobic parts of the peptide may interact with hydrophobic parts of the matrix material and thus loose its ability to bind to the component, which is to be bound by the matrix. By coating the matrix with a polyalcohol, hydrophobic parts of the matrix material are made more hydrophilic, since hydrophobic parts of the polyalcohol interact with the hydrophobic parts of the matrix material and the hydrophilic parts of the polyalcohol are faced towards the peptide, thereby hindering the hydrophobic interaction between the peptide and the matrix material.

Before the matrix is used, it is rinsed with a physiological NaCl-solution to remove the polyalcohol. It is advantageous if the polyalcohol is biocompatible, i.e. non-toxic, in the event smaller residual amounts of the polyalcohol are left after rinsing.

Examples of biocompatible polyalchols are propane-1,2,3-triol, glucose, trehalose and a mixture thereof. Preferably, the polyalcohol is propane-1,2,3-triol, also known as glycerol or glycerin(e). Propane-1,2,3-triol is an endogenous non-toxic compound, making it especially suitable for coating of the matrix. Furthermore, it is a liquid at room temperature and is thus easy to handle. Further, since it is a liquid it will not evaporate or crystallize and is thus an especially effective humectant.

Another advantage of the presence of a polyalcohol-coating is that such a coating acts as a radical-scavenger, thereby protecting the matrix from by beta- and gamma-radiation, which may be used for sterilizing the matrix.

Preferably, the polyalcohol is used in an amount corresponding to up to 0.1 to 0.5 g/m² of the matrix.

The peptide is chosen such that it binds to the component, which is to be bound by the matrix.

The peptide may be a peptide that binds LPS. Preferably, the LPS-binding peptide is 4 to 40 amino acids long, more preferably 10 to 35 amino acids long, and most preferably 20 to 30 amino acids long. More preferably, the LPS-binding peptide is a peptide according to SEQ ID NO 1 or a peptide having at least 80%, 85%, 90%, 95% or 99% homology with the peptide according to SEQ ID NO 1.

The matrix may be sintered polyethylene, preferably functionalized with amino-groups as detailed above.

The pore size of the matrix may range from 1 μm to 500 μm, more preferably from 70 um to 170 μm, most preferred from 80 μm to 100 μm. When the body fluid does not contain any blood cells, the pore size may be 1 μm to 25 μm.

The matrix may have an active surface ranging from 0.5 cm² to 10 m², preferably 4 cm² to 6 m², as measured by the BET-method, measured either by nitrogen adsorption or mercury intrusion.

The linker may be covalently bound to a residue of an amino-group present in the matrix and covalently bound to a residue of an amino-group present in the peptide.

Preferably, the linker is covalently bound to a residue of an amino-group present in the matrix and covalently bound to a residue of a thiol-group present in the peptide.

The linker may be chosen from the group consisting of succinimidyl 4-(N-maleimido-methyl)cyclohexane-1-carboxylate (SMCC), sulfo-succinimidyl 4-(N-maleimidomethyl)-cyclohexane-1-carboxylate (Sulfo-SMCC), 4-((4-(cyanoethynyl)benzoyl)oxy)-2,3,5,6-tetrafluorobenzenesulfonate (CBTF), sulfo-4-((4-(cyanoethynyl)benzoyl)oxy)-2,3,5,6-tetra-fluorobenzenesulfonate (Sulfo-CBTF), maleimide-poly(ethylene glycol)-succinimidyl ester, poly(ethylene glycol)-diglycidyl ether and 1-4-butanediol diglycidyl ether. The nature of these linkers is detailed above. Preferably, the linker is SMCC, CBTF, maleimide-poly(ethylene glycol)-succinimidyl ester, poly(ethylene glycol)-diglycidyl ether or 1-4-butanediol diglycidyl ether. Most preferably, the linker is SMCC or CBTF.

One specific example of Matrix A has a porous structure and a peptide is covalently attached to the matrix via a linker; wherein the matrix is coated with propane-1,2,3-triol. The matrix is preferably sintered polyethylene functionalized with amino-groups as detailed above.

Another specific example of Matrix A has a porous structure and a peptide is covalently attached to the matrix via a linker; wherein the linker is covalently bound to a residue of an amino-group present in the matrix and covalently bound to a residue of an amino-group present in the peptide; and wherein the matrix is coated with propane-1,2,3-triol. The matrix is preferably sintered polyethylene functionalized with amino-groups as detailed above.

A further specific example of Matrix A has a porous structure and a peptide is covalently attached to the matrix via a linker; wherein the linker is covalently bound to a residue of an amino-group present in the matrix and covalently bound to a residue of a thiol-group present in the peptide; and wherein the matrix is coated with propane-1,2,3-triol. The matrix is preferably sintered polyethylene functionalized with amino-groups as detailed above.

Another specific example of Matrix A has a porous structure and a peptide is covalently attached to the matrix via a linker; wherein the linker is SMCC or CBTF; wherein the linker is covalently bound to a residue of an amino-group present in the matrix and covalently bound to a residue of a thiol-group present in the peptide; and wherein the matrix is coated with propane-1,2,3-triol. The matrix is preferably sintered polyethylene functionalized with amino-groups as detailed above.

Another specific example of Matrix A has a porous structure and a peptide is covalently attached to the matrix via a linker; wherein the linker is maleimide-poly(ethylene glycol)-succinimidyl ester, poly(ethylene glycol)-diglycidyl ether or 1-4-butanediol diglycidyl ether; wherein the linker is covalently bound to a residue of an amino-group present in the matrix and covalently bound to a residue of a thiol-group present in the peptide; and wherein the matrix is coated with propane-1,2,3-triol. The matrix is preferably sintered polyethylene functionalized with amino-groups as detailed above.

Another specific example of Matrix A has a porous structure and an LPS-binding peptide is covalently attached to the matrix via a linker; wherein the linker is covalently bound to a residue of an amino-group present in the matrix and covalently bound to a residue of an amino-group present in the peptide; and wherein the matrix is coated with propane-1,2,3-triol. The LPS-binding peptide is a peptide according to SEQ ID NO 1 or a peptide having at least 80%, 85%, 90%, 95% or 99% homology with the peptide according to SEQ ID NO 1. The matrix is preferably sintered polyethylene functionalized with amino-groups as detailed above.

Another specific example of Matrix A has a porous structure and an LPS -binding peptide is covalently attached to the matrix via a linker; wherein the linker is covalently bound to a residue of an amino-group present in the matrix and covalently bound to a residue of a thiol-group present in the peptide; and wherein the matrix is coated with propane-1,2,3-triol. The LPS-binding peptide is a peptide according to SEQ ID NO 1 or a peptide having at least 80%, 85%, 90%, 95% or 99% homology with the peptide according to SEQ ID NO 1. The matrix is preferably sintered polyethylene functionalized with amino-groups as detailed above.

Yet another specific example of Matrix A has a porous structure and an LPS -binding peptide is covalently attached to the matrix via a linker; wherein the linker is SMCC or CBTF; wherein the linker is covalently bound to a residue of an amino-group present in the matrix and covalently bound to a residue of a thiol-group present in the peptide; and wherein the matrix is coated with propane-1,2,3-triol. The LPS -binding peptide is a peptide according to SEQ ID NO 1 or a peptide having at least 80%, 85%, 90%, 95% or 99% homology with the peptide according to SEQ ID NO 1. The matrix is preferably sintered polyethylene functionalized with amino-groups as detailed above.

Yet another specific example of Matrix A has a porous structure and an LPS -binding peptide is covalently attached to the matrix via a linker; wherein the linker is maleimide-poly(ethylene glycol)-succinimidyl ester, poly(ethylene glycol)-diglycidyl ether or 1-4-butanediol diglycidyl ether; wherein the linker is covalently bound to a residue of an amino-group present in the matrix and covalently bound to a residue of a thiol-group present in the peptide; and wherein the matrix is coated with propane-1,2,3-triol. The LPS-binding peptide is a peptide according to SEQ ID NO 1 or a peptide having at least 80%, 85%, 90%, 95% or 99% homology with the peptide according to SEQ ID NO 1. The matrix is preferably sintered polyethylene functionalized with amino-groups as detailed above.

Matrix B has a porous structure. A peptide is covalently attached to the matrix via a linker; wherein the linker is covalently bound to a residue of an amino-group present in the matrix and covalently bound to a residue of a thiol-group present in the peptide; and wherein the linker is a heterobifunctional cross-linker chosen from the group consisting of succinimidyl 4-(N-maleimidomethyl)cyclohexane-1-carboxylate (SMCC), sulfo-succinimidyl 4-(N-maleimidomethyl)cyclohexane-1-carboxylate (Sulfo-SMCC), 4-4-((4-(cyanoethynyl)benzoyl)-oxy)-2,3,5,6-tetrafluorobenzenesulfonate (CBTF), sulfo-4-((4-(cyanoethynyl)benzoyl)oxy)-2,3,5,6-tetrafluorobenzenesulfonate (Sulfo-CBTF), maleimide-poly (ethylene glycol)-succinimidyl ester, poly(ethylene glycol)-diglycidyl ether and 1-4-butanediol diglycidyl ether.

Such linkers have been shown to bind the peptide in a stable manner to the matrix.

Since the linker binds to two different functional groups, the risk of the improper binding of the peptide is reduced.

The linkers disclosed herein act as coupling agents, coupling the peptide to the matrix. In addition, the linkers also provide a means to create a distance between the matrix and the peptide, such that the availability of the peptide to the components present in the body fluid is increased, thereby increasing the number of binding sites for the component in the matrix. Preferably, the linker creates a distance from the surface of the matrix to the peptide of 6 carbon atoms or more.

In maleimide-poly(ethylene glycol)-succinimidyl ester, the poly(ethylene glycol)-moiety preferably comprises 2 to 20, such as 2 to 15, such as 2 to 8 poly(ethylene glycol)-moieties.

Poly(ethylene glycol) diglycidyl ether and 1-4-butanediol diglycidyl ether bind selectively to amino-groups at a pH-value of about 11, and to tiol-groups at a pH-value of about 8 to 9. Thus, by controlling the pH, the nature of the group to which these linkers bind, may be controlled.

In poly(ethylene glycol)-diglycidyl ether, the poly(ethylene glycol)-moiety preferably comprises 2 to 20, such as 2 to 15, such as 2 to 8 poly(ethylene glycol)-moieties. The poly(ethylene glycol)-diglycidyl ether may have a molecular weight of 200 to 2000 g/mol.

Preferably, the linker is SMCC, CBTF, maleimide-poly(ethylene glycol)-succinimidyl ester, poly(ethylene glycol)-diglycidyl ether or 1-4-butanediol diglycidyl ether. Most preferably, the linker is SMCC or CBTF.

The peptide is a peptide capable of binding to a specific component present in the body fluid.

The peptide may be a peptide that binds LPS. Preferably, the LPS-binding peptide is 4 to 40 amino acids long, more preferably 10 to 35 amino acids long, and most preferably 20 to 30 amino acids long. More preferably, the LPS-binding peptide is a peptide according to SEQ ID NO 1 or a peptide having at least 80%, 85%, 90%, 95% or 99% homology with the peptide according to SEQ ID NO 1.

The matrix may be sintered polyethylene, preferably functionalized with amino-groups as detailed above.

The pore size of the matrix may range from 1 μm to 500 μm, more preferably from 70 um to 170 μm, most preferred from 80 μm to 100 μm. When the body fluid does not contain any blood cells, the pore size may be 1 μm to 25 μm.

The matrix may have an active surface ranging from 0.5 cm² to 10 m², preferably 4 cm² to 6 m², as measured by the BET-method, measured either by nitrogen adsorption or mercury intrusion.

The matrix may be coated with a polyalcohol as discussed above. Preferably, the polyalcohol is chosen from the group comprising of propane-1,2,3-triol, glucose, trehalose and a mixture thereof. Most preferably, the polyalcohol is propane-1,2,3-triol.

Preferably, the polyalcohol is used in an amount corresponding to up to 0.1 to 0.5 g/m² of the matrix.

One specific example of Matrix B has a porous structure and a peptide is covalently attached to the matrix via a linker; wherein the linker is covalently bound to a residue of an amino-group present in the matrix and covalently bound to a residue of a thiol-group present in the peptide; and wherein the linker is SMCC or CBTF. The matrix is preferably sintered polyethylene functionalized with amino-groups as detailed above.

One specific example of Matrix B has a porous structure and a peptide is covalently attached to the matrix via a linker; wherein the linker is covalently bound to a residue of an amino-group present in the matrix and covalently bound to a residue of a thiol-group present in the peptide; and wherein the linker is maleimide-poly(ethylene glycol)-succinimidyl ester, poly(ethylene glycol)-diglycidyl ether or 1-4-butanediol diglycidyl ether. The matrix is preferably sintered polyethylene functionalized with amino-groups as detailed above.

Another specific example of Matrix B has a porous structure and an LPS-binding peptide is covalently attached to the matrix via a linker; wherein the linker is covalently bound to a residue of an amino-group present in the matrix and covalently bound to a residue of a thiol-group present in the peptide; and wherein the linker is SMCC or CBTF. The LPS-binding peptide is preferably a peptide according to SEQ ID NO 1 or a peptide having at least 80%, 85%, 90%, 95% or 99% homology with the peptide according to SEQ ID NO 1. The matrix is preferably sintered polyethylene functionalized with amino-groups as detailed above.

Another specific example of Matrix B has a porous structure and an LPS-binding peptide is covalently attached to the matrix via a linker; wherein the linker is covalently bound to a residue of an amino-group present in the matrix and covalently bound to a residue of a thiol-group present in the peptide; and wherein the linker is maleimide-poly(ethylene glycol)-succinimidyl ester, poly(ethylene glycol)-diglycidyl ether or 1-4-butanediol diglycidyl ether. The LPS-binding peptide is preferably a peptide according to SEQ ID NO 1 or a peptide having at least 80%, 85%, 90%, 95% or 99% homology with the peptide according to SEQ ID NO 1. The matrix is preferably sintered polyethylene functionalized with amino-groups as detailed above.

Yet another specific example of Matrix B has a porous structure and a peptide is covalently attached to the matrix via a linker; wherein the linker is covalently bound to a residue of an amino-group present in the matrix and covalently bound to a residue of a thiol-group present in the peptide; and wherein the linker is SMCC or CBTF and wherein the matrix is coated with a polyalcohol. The matrix is preferably sintered polyethylene functionalized with amino-groups as detailed above. The polyalcohol is preferably propane-1,2,3-triol.

Yet another specific example of Matrix B has a porous structure and a peptide is covalently attached to the matrix via a linker; wherein the linker is covalently bound to a residue of an amino-group present in the matrix and covalently bound to a residue of a thiol-group present in the peptide; and wherein the linker is maleimide-poly(ethylene glycol)-succinimidyl ester, poly(ethylene glycol)-diglycidyl ether or 1-4-butanediol diglycidyl ether and wherein the matrix is coated with a polyalcohol. The matrix is preferably sintered polyethylene functionalized with amino-groups as detailed above. The polyalcohol is preferably propane-1,2,3-triol.

Yet another specific example of Matrix B has a porous structure and an LPS -binding peptide is covalently attached to the matrix via a linker; wherein the linker is covalently bound to a residue of an amino-group present in the matrix and covalently bound to a residue of a thiol-group present in the peptide; and wherein the linker is SMCC or CBTF and wherein the matrix is coated with a polyalcohol. The matrix is preferably sintered polyethylene functionalized with amino-groups as detailed above. The LPS-binding peptide is preferably a peptide according to SEQ ID NO 1 or a peptide having at least 80%, 85%, 90%, 95% or 99% homology with the peptide according to SEQ ID NO 1. The polyalcohol is preferably propane-1,2,3-triol.

Yet another specific example of Matrix B has a porous structure and an LPS -binding peptide is covalently attached to the matrix via a linker; wherein the linker is covalently bound to a residue of an amino-group present in the matrix and covalently bound to a residue of a thiol-group present in the peptide; and wherein the linker is maleimide-poly(ethylene glycol)-succinimidyl ester, poly(ethylene glycol)-diglycidyl ether or 1-4-butanediol diglycidyl ether and wherein the matrix is coated with a polyalcohol. The matrix is preferably sintered polyethylene functionalized with amino-groups as detailed above. The LPS-binding peptide is preferably a peptide according to SEQ ID NO 1 or a peptide having at least 80%, 85%, 90%, 95% or 99% homology with the peptide according to SEQ ID NO 1. The polyalcohol is preferably propane-1,2,3-triol.

Matrix C has a porous structure. A peptide according to SEQ ID NO 1 or a peptide having at least 80%, 85%, 90%, 95% or 99% homology with the peptide according to SEQ ID NO 1 is covalently attached to the matrix via a linker; wherein the linker is covalently bound to a residue of an amino-group present in the matrix and covalently bound to a residue of a thiol-group present in the peptide.

The cross-linker may be chosen from the group consisting of succinimidyl 4-(N-maleimidomethyl)cyclohexane-1-carboxylate (SMCC), sulfo-succinimidyl 4-(N-maleimidomethyl)cyclohexane-1-carboxylate (Sulfo-SMCC), 4-((4-(cyanoethynyl)benzoyl)oxy)-2,3,5,6-tetrafluorobenzenesulfonate (CBTF), sulfo-4-((4-(cyanoethynyl)benzoyl)oxy)-2,3,5,6-tetrafluorobenzenesulfonate (Sulfo-CBTF), maleimide-poly(ethylene glycol)-succinimidyl ester, poly(ethylene glycol)-diglycidyl ether and 1-4-butanediol diglycidyl ether; preferably wherein the linker is SMCC, CBTF, maleimide-poly(ethylene glycol)-succinimidyl ester, poly(ethylene glycol)-diglycidyl ether or 1-4-butanediol diglycidyl ether; most preferably wherein the linker is SMCC or CBTF.

The matrix may be coated with a polyalcohol as discussed above. Preferably, the polyalcohol is chosen from the group comprising of propane-1,2,3-triol, glucose, trehalose and a mixture thereof. More preferably, the polyalcohol is propane-1,2,3-triol.

Preferably, the polyalcohol is used in an amount corresponding to up to 0.1 to 0.5 g/m² of the matrix.

The matrix may be sintered polyethylene, preferably functionalized with amino-groups as detailed above.

The pore size of the matrix may range from 1 μm to 500 μm, more preferably from 70 um to 170 μm, most preferred from 80 μm to 100 μm. When the body fluid does not contain any blood cells, the pore size may be 1 μm to 25 μm.

The matrix may have an active surface ranging from 0.5 cm² to 10 m², preferably 4 cm² to 6 m², as measured by the BET-method, measured either by nitrogen adsorption or mercury intrusion.

One specific example of Matrix C has a porous structure and a peptide according to SEQ ID NO 1 or a peptide having at least 80%, 85%, 90%, 95% or 99% homology with the peptide according to SEQ ID NO 1 is covalently attached to the matrix via a linker; wherein the linker is covalently bound to a residue of an amino-group present in the matrix and covalently bound to a residue of a thiol-group present in the peptide. The matrix is preferably sintered polyethylene functionalized with amino-groups as detailed above.

Another specific example of Matrix C has a porous structure and a peptide according to SEQ ID NO 1 or a peptide having at least 80%, 85%, 90%, 95% or 99% homology with the peptide according to SEQ ID NO 1 is covalently attached to the matrix via a linker; wherein the linker is covalently bound to a residue of an amino-group present in the matrix and covalently bound to a residue of a thiol-group present in the peptide; chosen from the group consisting of succinimidyl 4-(N-maleimidomethyl)cyclohexane-1-carboxylate (SMCC), sulfo-succinimidyl 4-(N-maleimidomethyl)cyclohexane-1-carboxylate (Sulfo-SMCC), 4-((4-(cyanoethynyl)benzoyl)oxy)-2,3,5,6-tetrafluorobenzenesulfonate (CBTF), sulfo-4-((4-(cyanoethynyl)benzoyl)oxy)-2,3,5,6-tetrafluorobenzenesulfonate (Sulfo-CBTF), maleimide-poly(ethylene glycol)-succinimidyl ester, poly(ethylene glycol)-diglycidyl ether and 1-4-butanediol diglycidyl ether. The nature of these linkers is detailed above. Preferably, the linker is SMCC, CBTF, maleimide-poly(ethylene glycol)-succinimidyl ester, poly(ethylene glycol)-diglycidyl ether or 1-4-butanediol diglycidyl ether. Most preferably, the linker is SMCC or CBTF. The matrix is preferably sintered polyethylene functionalized with amino-groups as detailed above.

Yet another specific example of Matrix C has a porous structure and a peptide according to SEQ ID NO 1 or a peptide having at least 80%, 85%, 90%, 95% or 99% homology with the peptide according to SEQ ID NO 1 is covalently attached to the matrix via a linker; wherein the linker is covalently bound to a residue of an amino-group present in the matrix and covalently bound to a residue of a thiol-group present in the peptide; and wherein the matrix is coated with a polyalcohol. The polyalcohol is preferably propane-1,2,3-triol, glucose, trehalose or a mixture thereof. Preferably, the polyalcohol is propane-1,2,3-triol, also known as glycerol or glycerin(e). The matrix is preferably sintered polyethylene functionalized with amino-groups as detailed above. The linker is preferably chosen from the group consisting of succinimidyl 4-(N-maleimidomethyl)cyclohexane-1-carboxylate (SMCC), sulfo-succinimidyl 4-(N-maleimidomethyl)cyclohexane-1-carboxylate (Sulfo-SMCC), 4-4-(4-(cyanoethynyl)-benzoylloxy)-2,3,5,6-tetrafluorobenzenesulfonate (CBTF), sulfo-4-4-(4-(cyanoethynyl)-benzoyl)oxy)-2,3,5,6-tetrafluorobenzenesulfonate (Sulfo-CBTF), maleimide-poly(ethylene glycol)-succinimidyl ester, poly(ethylene glycol)-diglycidyl ether and 1-4-butanediol diglycidyl ether. The nature of these linkers is detailed above. More preferably, the linker is SMCC, CBTF, maleimide-poly(ethylene glycol)-succinimidyl ester, poly(ethylene glycol)-diglycidyl ether or 1-4-butanediol diglycidyl ether. Most preferably, the linker is SMCC or CBTF.

Method for Manufacturing a Matrix for Selective Binding of at Least One Component from a Body Fluid

The present disclosure also relates to a method for manufacturing a matrix for selective binding of at least one component from a body fluid. The method comprising the steps of a) providing a matrix comprising primary amino-groups; b) covalently attaching a peptide to the matrix via a linker to provide a matrix comprising a covalently attached peptide; c) adding a polyalcohol to the matrix comprising a covalently attached peptide; and d) subjecting the matrix obtained after step c) to irradiation.

The linker may be a linker that covalently binds to a residue of an amino-group present in the matrix and covalently binds to a residue of a thiol-group present in the peptide. Preferably, the linker is chosen from the group consisting of succinimidyl 4-(N-maleimido-methyl)cyclohexane-1-carboxylate (SMCC), sulfo-succinimidyl 4-(N-maleimidomethyl)-cyclohexane-1-carboxylate (Sulfo-SMCC), 4-((4-(cyanoethynyl)benzoyl)oxy)-2,3,5,6-tetrafluorobenzenesulfonate (CBTF), sulfo-4-((4-(cyanoethynyl)benzoyl)oxy)-2,3,5,6-tetra-fluorobenzenesulfonate (Sulfo-CBTF), maleimide-poly(ethylene glycol)-succinimidyl ester, poly(ethylene glycol)-diglycidyl ether and 1-4-butanediol diglycidyl ether. The nature of these linkers is detailed above. Preferably, the linker is SMCC, CBTF, maleimide-poly(ethylene glycol)-succinimidyl ester, poly(ethylene glycol)-diglycidyl ether or 1-4-butanediol diglycidyl ether. Most preferably, the linker is SMCC or CBTF.

Step b) preferably comprises the steps of b1) coupling of the cross-linker to amino-groups on the surface of the matrix, b2) removing uncoupled cross-linker, and b3) coupling of peptide to the cross-linker.

Preferably, step b) is carried out in solutions having an ion-strength of 0.05-0.2 M and a pH 5-8.

The peptide may be an LPS-binding peptide. Preferably, the LPS-binding peptide is 4 to 40 amino acids long, more preferably 10 to 35 amino acids long, and most preferably 20 to 30 amino acids long. More preferably, the LPS-binding peptide is a peptide according to SEQ ID NO 1 or a peptide having at least 80%, 85%, 90%, 95% or 99% homology with the peptide according to SEQ ID NO 1.

The polyalcohol may be propane-1,2,3-triol, glucose, trehalose or a mixture thereof. Preferably, the polyalcohol is propane-1,2,3-triol, also known as glycerol or glycerin(e).

The polyalcohol may be used in an amount corresponding to up to 0.1 to 0.5 g/m² of the matrix.

A matrix comprising primary amino-groups may be provided as described above or as known in the art.

The matrix may be sintered polyethylene, preferably functionalized with amino-groups as detailed above.

The pore size of the matrix may range from 1 μm to 500 μm, more preferably from 70 um to 170 μm, most preferred from 80 μm to 100 μm. When the body fluid does not contain any blood cells, the pore size may be 1 μm to 25 μm.

The matrix may have an active surface ranging from 0.5 cm² to 10 m², preferably 4 cm² to 6 m², as measured by the BET-method, measured either by nitrogen adsorption or mercury intrusion.

The step of irradiation may be carried out as irradiating the matrix by beta- or gamma-radiation.

In a specific example of a method for manufacturing a matrix for selective binding of at least one component from a body fluid, the matrix is sintered polyethylene functionalized with amino-groups as detailed above, and the polyalcohol is propane-1,2,3-triol.

In another specific example of a method for manufacturing a matrix for selective binding of at least one component from a body fluid, the matrix is sintered polyethylene functionalized with amino-groups as detailed above, the LPS-binding peptide is a peptide according to SEQ ID NO 1 or a peptide having at least 80%, 85%, 90%, 95% or 99% homology with the peptide according to SEQ ID NO 1, and the polyalcohol is propane-1,2,3-triol.

In yet another specific example of a method for manufacturing a matrix for selective binding of at least one component from a body fluid, the matrix is sintered polyethylene functionalized with amino-groups as detailed above, the linker is SMCC or CBTF, and the polyalcohol is propane-1,2,3-triol.

In yet another specific example of a method for manufacturing a matrix for selective binding of at least one component from a body fluid, the matrix is sintered polyethylene functionalized with amino-groups as detailed above, the linker is maleimide-poly(ethylene glycol)-succinimidyl ester, poly(ethylene glycol)-diglycidyl ether or 1-4-butanediol diglycidyl ether, and the polyalcohol is propane-1,2,3-triol.

In a further specific example of a method for manufacturing a matrix for selective binding of at least one component from a body fluid, the matrix is sintered polyethylene functionalized with amino-groups as detailed above, the LPS-binding peptide is a peptide according to SEQ ID NO 1 or a peptide having at least 80%, 85%, 90%, 95% or 99% homology with the peptide according to SEQ ID NO 1, the linker is SMCC or CBTF, and the polyalcohol is propane-1,2,3-triol.

In yet a further specific example of a method for manufacturing a matrix for selective binding of at least one component from a body fluid, the matrix is sintered polyethylene functionalized with amino-groups as detailed above, the LPS-binding peptide is a peptide according to SEQ ID NO 1 or a peptide having at least 80%, 85%, 90%, 95% or 99% homology with the peptide according to SEQ ID NO 1, the linker is maleimide-poly(ethylene glycol)-succinimidyl ester, poly(ethylene glycol)-diglycidyl ether or 1-4-butanediol diglycidyl ether, and the polyalcohol is propane-1,2,3-triol.

Method for Selectively Binding and Separating at Least One Component from a Body Fluid

The present disclosure also relates to a method for selectively binding and separating at least one component from a body fluid, comprising the step of passing a body fluid through a matrix as disclosed herein or through a matrix manufactured as disclosed herein, whereby said at least one component binds to the peptide bound covalently bound to the matrix.

The body fluid may be whole blood, plasma or cerebrospinal fluid.

Preferably, the body fluid is whole blood.

The component, which is to be selectively bound, may be an endogenous component, i.e. a component produced by the patient whose body fluid, such as whole blood, is to be passed through the matrix.

The component, which is to be selectively bound, may be an exogenous component, i.e. a component that is not produced by the patient whose body fluid, such as whole blood, is to be passed through the matrix. Examples of such components may be a toxic component produced by infectious agent, such as from a bacterium, a virus or a fungus.

More specifically, the component may be derived from a bacterium.

A specific example of a component, which is to be bound by a matrix disclosed herein, is LPS produced by Gram-negative bacteria.

Thus, preferred examples of the method relate to the selective binding of LPS and to the separation of toxic LPS from the body fluid, especially from the blood, of a patient suffering from sepsis caused by Gram-negative bacteria. In such cases, the peptide bound to the matrix is an LPS-binding peptide, preferably, an LPS-binding peptide which is 4 to 40 amino acids long, more preferably 10 to 35 amino acids long, and most preferably 20 to 30 amino acids long, more preferably a peptide according to SEQ ID NO 1 or a peptide having at least 80%, 85%, 90%, 95% or 99% homology with the peptide according to SEQ ID NO 1.

Device for Selective Binding and Separation of at Least One Component from a Body Fluid

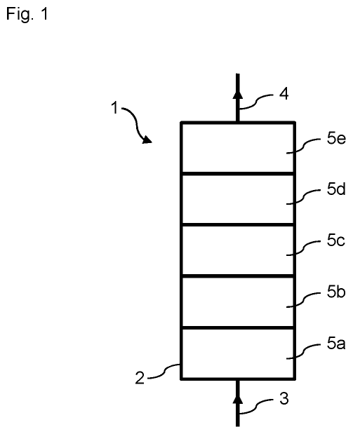

The present disclosure also relates to a device for selective binding and separation of at least one component from a body fluid. The device 1 as shown in FIG. 1 comprises a housing 2, an inlet 3, an outlet 4 and a first matrix 5, wherein the first matrix is a matrix according to the present disclosure or a matrix manufactured by the method according to the present disclosure.

A device 1 comprises a housing 2, the housing (or cartridge) of the device being integrated into a closed circulation, in which whole blood or a body fluid as described above, is circulated by means of a pump. In the housing 2 at least one separation matrix 5a, 5b, 5c, 5d, 5e is arranged, each intended to selectively remove one component from whole blood or from other exemplified body fluids. The housing 2 is provided with an inlet 3 and an outlet 4, the sites of which are of no importance as long as an adequate flow is obtained within the separation matrix(matrices) and the housing. Preferably, the pump is arranged upstream the inlet 3.

In this way a device is obtained which can maintain flow rates from 5 ml/h to 6 000 ml/min without a significant pressure drop. When applied extracorporeally, a line pressure of not more than 300 mm Hg from pump to cannula is obtained even at very high flow rates.

The rigid integral separation matrix can be produced in different shapes to be used in the inventive method. It can for example be designed as a disk, a rod, a cylinder, a ring, a sphere, a tube, a hollow tube, a flat sheet, or other moulded shapes.

Since the flow within each separation matrix is dependent on its porosity, the contact time of the components in blood or a body fluid with the active surface can be controlled. Furthermore, a desired flow gradient can be created within a separation device by changing the porosity and configuration of the individual separation matrices therein.

In some embodiments, more than one matrix is used. In such embodiments, the separation matrices can have the same or different porosities. Further, the peptides bound to the matrices may be the same or different. Thus, depending on the chosen matrices, one or several different components may be removed from the body fluid. The separation matrices are preferably integrated with the housings (each having an inlet 3 and an outlet 4) in order to ensure that no liquid or components therein are prevented from entering the matrix or matrices, i.e. being excluded therefrom.

In one example of a device according to the present disclosure, the peptide bound to the matrix is an LPS-binding peptide, preferably a peptide according to SEQ ID NO 1 or a peptide having at least 80%, 85%, 90%, 95% or 99% homology with the peptide according to SEQ ID NO 1. Such a device is used to selectively remove LPS from the blood (or other body fluid) of a patient suffering from sepsis caused by Gram-negative bacteria.

Use of a matrix as disclosed herein, of a matrix manufactured by the method disclosed herein or of a device disclosed herein for selectively binding and separating at least one component from a body fluid.

The present disclosure also relates to the use of a matrix according to the present disclosure, of a matrix manufactured by a method according to the present disclosure or of a device according of the present disclosure.

Such a matrix or device may be used in the treatment of a patient suffering from sepsis caused by Gram-negative bacteria. In such cases, the matrix used comprises an LPS-binding peptide. Preferably, the LPS-binding peptide is 4 to 40 amino acids long, more preferably 10 to 35 amino acids long, and most preferably 20 to 30 amino acids long. More preferably, the peptide is a peptide according to SEQ ID NO 1 or a peptide having at least 80%, 85%, 90%, 95% or 99% homology with the peptide according to SEQ ID NO 1.

Amino Acid Sequence

The sequence originates from horseshoe crab (Limulus polyphemus).

SEQ ID NO 1:
HAEHKVKIKVKQKYGQFPQGTEVTYTC

1. A matrix for selective binding of at least one component from a body fluid, the matrix comprising:
a porous structure;
a linker;
a peptide covalently attached via the linker; and
a coating of a polyalcohol.

2. The matrix according to claim 1, wherein the polyalcohol is one of propane-1,2,3-triol, glucose, trehalose and a mixture thereof.

3. The matrix according to claim 1, wherein the peptide is an LPS-binding peptide.

4. The matrix according to claim 1, wherein the peptide is one of:
a peptide according to SEQ ID NO 1; and
a peptide having at least 80%, homology with a peptide according to SEQ ID NO 1.

5. The matrix according to claim 1, further comprising a sintered polyethylene.

6. The matrix according to claim 1, further comprising an amino-group, wherein:
the peptide includes a thiol-group; and
the linker is covalently bound to a residue of the amino-group and covalently bound to a residue of the thiol-group of the peptide.

7. The matrix according to claim 1, wherein:
the linker is a heterobifunctional cross-linker; and
the heterobifunctional cross-linker is one of succinimidyl 4-(N-maleimidomethyl)cyclohexane-1-carboxylate (SMCC), sulfo-succinimidyl 4-(N-maleimidomethyl)cyclohexane-1 -carboxylate (Sulfo-SMCC), 4-((4-(cyanoethynyl)benzoyl)oxy)-2,3,5,6 -tetrafluorobenzenesulfonate (CBTF), sulfo-4-((4-(cyanoethynyl)benzoyl)oxy)-2,3,5,6-tetrafluorobenzenesulfonate (Sulfo-CBTF), maleimide-poly(ethylene glycol)-succinimidyl ester, poly(ethylene glycol)-diglycidyl ether, and 1-4-butanediol diglycidyl ether.

8. A matrix for selective binding of at least one component from a body fluid, the matrix comprising:
a porous structure;
a linker;
a peptide covalently attached via the linker, the peptide including a thiol-group; and
an amino-group;
wherein the linker is covalently bound to a residue of the amino-group and covalently bound to a residue of the thiol-group of the peptide;
wherein the linker is a heterobifunctional cross-linker; and
wherein the heterobifunctional cross-linker is one of succinimidyl 4-(N-maleimidomethyl)cyclohexane-1-carboxylate (SMCC), sulfo-succinimidyl 4-(N-maleimidomethyl)cyclohexane-1-carboxylate (Sulfo-SMCC), 4-((4-(cyanoethynyl)benzoyl)oxy)-2,3,5,6 -tetrafluoro-benzenesulfonate (CBTF), sulfo-4-((4-(cyanoethynyl)benzoyl)oxy)-2,3,5,6 -tetra-fluorobenzenesulfonate (Sulfo-CBTF), maleimide-poly(ethylene glycol)-succinimidyl ester, poly(ethylene glycol)-diglycidyl ether and 1-4-butanediol diglycidyl ether.

9. The matrix according to claim 8, wherein the heterobifunctional cross-linker is one of SMCC, CBTF, maleimide-poly(ethylene glycol)-succinimidyl ester, poly(ethylene glycol)-diglycidyl ether, and 1-4-butanediol diglycidyl ether.

10. The matrix according to claim 8, wherein the peptide is one of:
a peptide according to SEQ ID NO 1; and
a peptide having at least 80% homology with a peptide according to SEQ ID NO 1.

11. The matrix according to claim 8, further comprising a coating of a polyalcohol.

12. The matrix according to claim 8, further comprising a sintered polyethylene.

13. A matrix for selective binding of lipopolysaccharide (LPS) from a body fluid, the matrix comprising:
a porous structure;
a linker;
a peptide covalently attached via the linker, the peptide including a thiol-group; and
an amino-group;
wherein the peptide is one of (i) a peptide according to SEQ ID NO 1 and (ii) a peptide having at least 80% homology with a peptide according to SEQ ID NO 1; and
wherein the linker is covalently bound to a residue of the amino-group and covalently bound to a residue of the thiol-group of the peptide.

14. The matrix according to claim 13, wherein the linker is one of succinimidyl 4-(N-maleimidomethyl)cyclohexane-1-carboxylate (SMCC), sulfo-succinimidyl 4-(N-maleimidomethyl)cyclohexane-1-carboxylate (Sulfo-SMCC), 4-((4-(cyanoethynyl)benzoyl)oxy)-2,3,5,6-tetrafluoro-benzenesulfonate (CBTF), sulfo-4-((4-(cyanoethynyl)benzoyl)oxy)-2,3,5,6-tetra-fluorobenzenesulfonate (Sulfo-CBTF), maleimide-poly(ethylene glycol)-succinimidyl ester, poly(ethylene glycol)-diglycidyl ether and 1-4-butanediol diglycidyl ether.

15. The matrix according to claim 13, further comprising a coating of a polyalcohol.

16. The matrix according to claim 13, further comprising a sintered polyethylene.

17. A method for manufacturing a matrix for selective binding of at least one component from a body fluid, the method comprising:
providing a first matrix including a plurality of primary amino-groups;
covalently attaching a peptide to the first matrix via a linker to provide a second matrix including a covalently attached peptide;
adding a polyalcohol to the second matrix to provide a third matrix; and
subjecting the third matrix to irradiation.

18. The method according to claim 17, wherein:
the linker is a heterobifunctional cross-linker; and
the heterobifunctional cross-linker is one of succinimidyl 4-(N-maleimidomethyl)cyclohexane-1-carboxylate (SMCC), sulfo-succinimidyl 4-(N-maleimidomethyl)cyclohexane-1-carboxylate (Sulfo-SMCC), 4-((4-(cyano-ethynyl)benzoyl)oxy)-2,3,5,6-tetrafluorobenzenesulfonate (CBTF), sulfo-4-((4-(cyanoethynyl)benzoyl)oxy)-2,3,5,6-tetrafluorobenzenesulfonate (Sulfo-CBTF), maleimide-poly(ethylene glycol)-succinimidyl ester, poly(ethylene glycol)-diglycidyl ether and 1-4-butanediol diglycidyl ether.

19. The method according to claim 17, wherein the peptide is one of:
a peptide according to SEQ ID NO 1; and
a peptide having at least 80% homology with a peptide according to SEQ ID NO 1.

20. The method according to claim 17, wherein the polyalcohol is one of propane-1,2,3-triol, glucose, trehalose, or a mixture thereof.

21. The method according to claim 17, further comprising a sintered polyethylene.

22. A method for selectively binding and separating at least one component from a body fluid, comprising:
passing the body fluid through the matrix according to claim 1; and
wherein the at least one component binds to the peptide covalently bound to the matrix.

23. A device for selective binding and separation of at least one component from a body fluid, the device comprising a housing, an inlet, an outlet, and at least one matrix, according to claim 1.

24. The device according to claim 23, wherein the at least one matrix includes a first matrix and a second matrix.

25. A method of using the matrix according to claim 1, comprising selectively binding and separating at least one component from a body fluid.
